(19) United States
(12) Patent Application Publication
(10) Pub. No.: US 2021/0219834 A1
(43) Pub. Date: Jul. 22, 2021

(54) VIDEO LARYNGOSCOPE THAT INCLUDES A BASE FOR A BLADE WITH ADJUSTABLE ANGULAR POSITION

(71) Applicant: Héctor De Jesús Velez Rivera, Zapopan (MX)

(72) Inventor: Héctor De Jesús Velez Rivera, Zapopan (MX)

(21) Appl. No.: 17/045,871

(22) PCT Filed: Apr. 9, 2018
(86) PCT No.: PCT/IB2018/052434

§ 371 Date: Mar. 30, 2021

(51) Int. Cl. A61B 1/267; A61B 1/05

(52) U.S. Cl. CPC A61B 1/267; A61B 1/05

(57) ABSTRACT

The invention describes a videolaryngoscope that comprises: a hollow handle with a proximal end and a distal end; a mounting base for a video laryngoscope blade, the mounting base being rotatably connected to the distal end of the handle and having a distal end; a videocamera mounted on the distal end of the mounting base for the video laryngoscope blade; a head mounted on the proximal end of the handle; a screen housed in the head with rotary movement relative to the proximal end of the handle and angular movement from top to bottom, which communicates operationally with the video camera; a coil receiving a charge by induction, located in the head; a rechargeable internal power source operationally connected with the charge-receiving coil; and a charging base that couples with the head.

### FIELD OF THE INVENTION

The instant invention refers to technics used in the design and construction of apparatuses and devices for medical use and, more particularly, it is related to a videolaryngoscope having a handle and a base for a blade, the base being rotably mounted to the handle such that said base for a blade has an adjustable angular position for varying its tilt angle relative to the handle and, so that, being used in the largest number of patients with both normal and special anthropometric characteristics. The videolaryngoscope further comprises elements of power recharge by induction when it is not in use.

### BACKGROUND OF THE INVENTION

The laryngoscope is an instrument used for directly visualizing the larynx in order to perform an endotracheal intubation for delivering anesthetics. The laryngoscope basically comprises two main portions, specifically, a handle for handling the laryngoscope and a blade mounted to the handle which is inserted into the larynx of the patient and it functions to separate the tongue and the epiglottis. At the end of the blade, usually there is a light source (a small light bulb or a lightpoint of optical fiber originating in the handle) to make more visible the larynx.

The blade of a videolaryngoscope may be reusable, in which case the same must be sterilized after each use or the blade may be disposable, in this last case, the laryngoscopes incorporate a blade base, that is a support in which the laryngoscope blade is coupled.

Over time and with the miniaturization of electronic components, the inclusion of cameras at the end of the laryngoscope blade has been made in order to visualize the larynx through video screens operationally connected to the camera, said devices are actually already known in a general manner as videolaryngoscopes.

Although a wide variety of videolaryngoscopes have been developed, there are medical requirements and operative problems thereof which have not been sufficiently addressed in the state of the art. One of such problems is related to the fact that the base for the laryngoscope blade is fixed, i.e., the base only has one position of use or tilt, nevertheless the patients have anthropometric proportions very different therebetween, as for example there are patients with too long or too short necks, or robust patients with a great volume in their rib cage hindering the handling of the videolaryngoscope above their chest, thus delaying and complicating the process of intubation.

Another problem with the videolaryngoscopes is related with the form in which they are powered via cables or voluminous batteries avoiding the location thereof in the more proper position both for the physician and the patient in order to introduce the videolaryngoscope blade.

### SUMMARY OF THE INVENTION

In order to overcome the problems of the state of the art, it has been developed a videolaryngoscope comprising: a handle preferably hollow with a proximal end and a distal end; another element of the invention is a mounting base for a videolaryngoscope blade, the mounting base is rotatably connected to the distal end of the handle for varying its angular position from top to bottom relative to the handle by pivoting at the distal end, additionally, the base for a base has a distal end in which a videocamera is mounted.

Likewise, the instant invention comprises a head rotatably mounted to the proximal end of the handle. Furthermore, there is a screen housed in the head and which is operationally communicated to the videocamera which is in the base of the mounting blade, in such way, the screen projects the images being captured by the videocamera.

An important feature of instant invention is the inclusion of an induction power receiving coil located in the head; in addition, there is an internal power storing source being rechargeable, the storing source is operationally connected to the power receiving coil. Finally, there is a recharge base being coupled to the head and including an induction power receiving coil, such that when the head is coupled to the recharge base, the internal power source is recharged such that when the videolaryngoscope is required, the internal power source provides power to the electronic components of the videolaryngoscope.

In the preferred embodiment of instant invention, the internal power storing source is a battery located inside the handle or the head, more preferably, the battery is housed inside the handle.

The head and the recharge base include cooperating magnetic elements to maintain the head and the recharge base in contact and without moving in order to align the generating coil of the recharge base to the receiving coil of the head and in such way efficiently recharging the internal battery by receiving the power generated by induction.

Regarding the structure of the head, in a preferred embodiment of the invention, the head comprises a housing with a frontal face and a rear face; wherein the screen is located in the frontal face; and, the power receiving coil is located in the interior of the head and next to the rear face. It is preferred the screen is immersed relative to the plane outermost of the frontal face, thus avoiding the scratch of the screen when the head is coupled in the recharge base. In this particular, it is preferably the screen is a touch screen.

Even more, the head is designed such that the frontal face has the superficial area necessary for maintaining the videolaryngoscope in vertical position and supporting all the weight when the head is recharging by coupling to the recharge base.

Regarding the aforementioned, the recharging base comprises a housing in which a power generating coil is located; a controller located inside the housing and operationally communicated to the coil for regulating the time and intensity of charge; another portion of the housing is a cavity wherein the head is coupled.

Regarding the head in which the screen is located, the head is mounted to the handle in the following manner: 1) it is rotably mounted to the proximal end of the handle, i.e., it has a rotation point in the proximal end and 2) it is mounted in a rotatory manner and separated from the proximal end for pivoting from top to bottom relative to the handle. In such way, the head and therefore the screen has movement freedom for i) positioning the head at any desired angle and to view the introduction of the blade through the screen; ii) being able to move the head in different directions and iii) avoiding the head interferes with the patient's body when introducing the videolaryngoscope blade.

In light of the aforementioned, the mounting base for the laryngoscope blade has at least one angular position that may be blocked for maintaining the mounting blade in a desired angle relative to the longitudinal axis of the handle.

In the preferred embodiment of the invention, in the distal end of the handle a rotatory mechanism for the mounting base of the blade is provided and thus varying its tilt relative to the handle, the rotatory mechanism is housed in the handle, also there is a blocking element of the rotatory mechanism for maintaining the mounting base in the desired angle relative to the handle.

### BRIEF DESCRIPTION OF THE FIGURES

The novel aspects that characterize the instant invention will be now established, particularly in the attached claims. However, the invention together with other objects and advantages thereof should be better understood in the below detailed description of some preferred embodiments of the invention, when reading together with the figures, which disclose:

FIG. 1 is a front and right lateral perspective of a videolaryngoscope constructed according the principles of instant invention.

FIG. 2 is a frontal view of the videolaryngoscopes of FIG. 1.

FIG. 3 is a right lateral view of the videolaryngoscope of FIG. 1.

FIG. 4 is an exploded view of the videolaryngoscope of FIG. 1.

FIG. 5 is a cross section view along line A-A′ of FIG. 2.

FIGS. 6A, 6B and 6C show the mounting of the head to the handle.

FIGS. 7A, 7B and 7C show a movement sequence for releasing and blocking the base for the laryngoscope blade.

FIG. 8 is an exploded view of the recharge base for the videolaryngoscope.

FIG. 9 is a lateral view of the videolaryngoscope and the recharge base being coupled.

### DETAILED DESCRIPTION OF THE PREFERRED EMBODIMENTS OF THE INVENTION

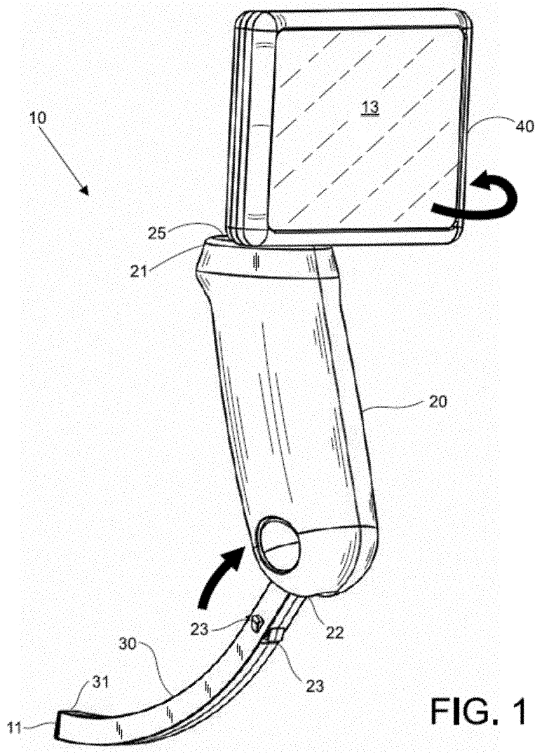

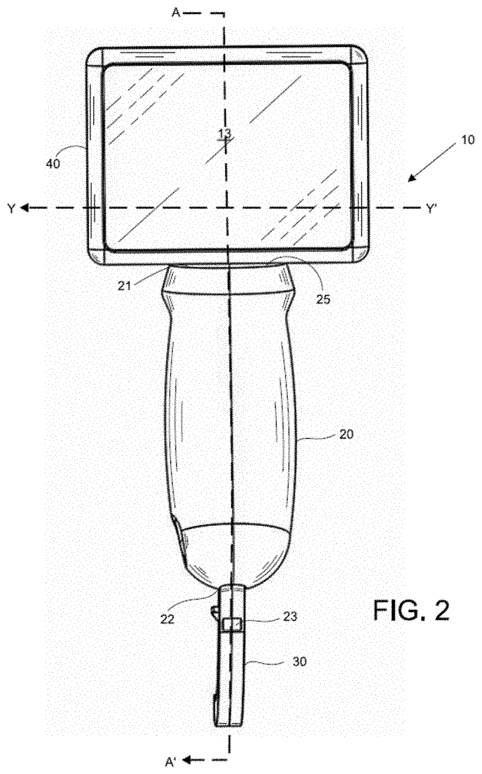

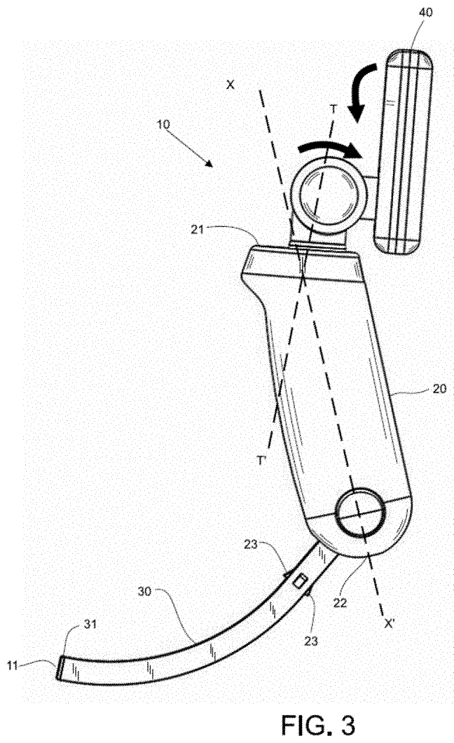

Referring to FIGS. 1 to 3 of the attached drawings, there is shown a videolaryngoscope 10 constructed according to the principles of instant invention, such embodiment must be considered only as illustrative and not as limitative of the invention.

In FIGS. 1 to 3, it is observed a videolaryngoscope 10 that comprises: a handle 20 having an ergonomic shape, i.e., it conforms to the hand palm, the handle 20 is preferably hollow for housing different components of the videolaryngoscope which will be disclosed below, the handle has a proximal end 21 and a distal end 22. The proximal end 21 is that located nearer the user that manipulates the videolaryngoscope and the distal end 22 is that far away of the user and nearer to the patient.

The handle 20 is provided with a mounting base 30 for a videolaryngoscope blade, the mounting base 30 is connected to the distal end 22 of the handle 20 in a rotatory manner for varying its angular position relative to the axial axis of the handle, i.e., the mounting base 30 is downwardly or upwardly movable by pivoting on the handle 20. The mounting base 30 has a distal end 21, in which a video camera 11 is mounted. The mounting base 30 for the blade has quadrangular shape in cross section and it has cooperating projections 23 for coupling therein a videolaryngoscope blade, such as a Macintosh or Miller blade.

Likewise, the instant invention comprises a head 40 rotably mounted to the proximal end 21 defined by an inclined flat wall 25. More particularly, the head 40 is rotably mounted and forwardly tilted on the T-T′ axis that is perpendicular to the inclined wall 25. That is, the head is forwardly moved a distance relative to the X-X′ axial axis of the handle. Even more, the head 40 is also rotably mounted on the Y-Y′ axis being transversal to the T-T′ axis; the Y-Y′ axis is upwardly separated relative to the inclined wall 25, i.e., the pivoting axis of the head is separated relative to the handle such that the head varies its tilt angle relative to the handle 20, i.e., such that the same is upwardly movable on the Y-Y′ axis according the preference of positioning of the medical staff.

The manner in which the head 40 is mounted to the handle 20 in order to move the head from top to bottom provides the advantage the head 40 may have a freedom of movement and positioning in the desired angle such that the head 40 does not interfere with the patient's body during the introduction of the videolaryngoscope blade into the patient, another advantage is allow the rotation and tilting the head to view the screen 13 in the best position for the medical staff.

Regarding the screen 13, the same is housed in the head 40 and it is operationally communicated to the video camera 11 that is located in the base for the blade 30 of the videolaryngoscope, in such way, once the videolaryngoscope blade is inside the patient's neck, the videocamera 11 captures images of the larynx which are transmitted to the screen 13 and they are easily observed by the user.

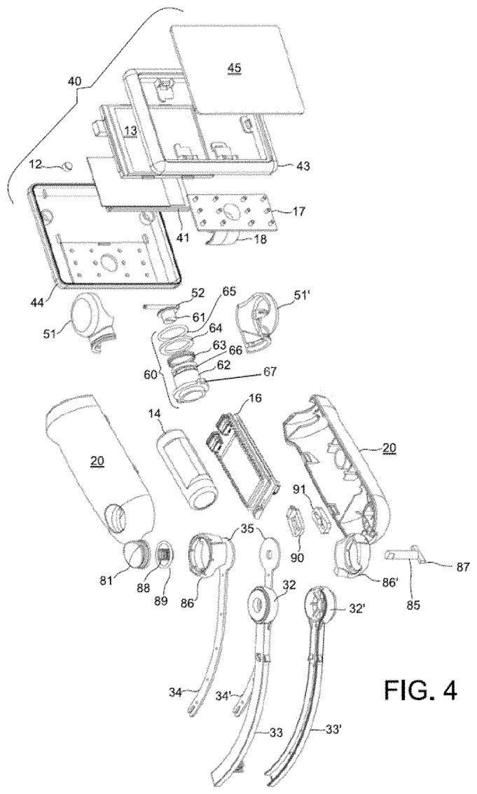

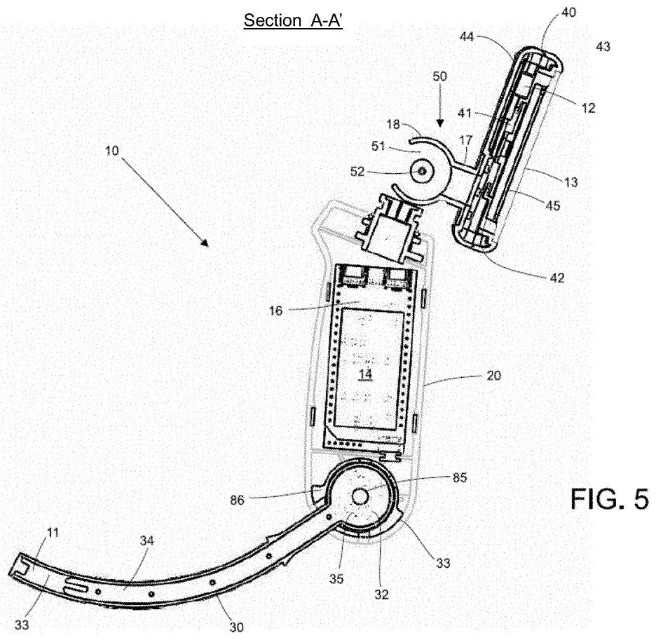

Now, reference is made to FIGS. 4 and 5 for mentioning that inside the head 40 there is an induction energy receiving coil 41; on the other hand, the handle 20 comprises two halves being coupled each to other, inside the handle there is a power storage source 14 being rechargeable and it operationally connected to the charge receiving coil 41, the functions of the videolaryngoscope are controlled via the integrated circuits board 16 also located in the handle 20 and next the power storage source 14.

In the preferred embodiment of the invention, the head 40 comprises a housing with a frontal face 43 and a rear face 44 being coupled therebetween for housing different elements as the screen 13 located in the frontal face 43, the screen is covered by a protection window 45; meanwhile the power receiving coil 41 is interiorly located in the head 40 and next to the rear face 44. It is preferably the screen is immersed relative to the outermost plan of the frontal face 43, thus avoiding screen 13 is damaged when resting in any surface to recharge the videolaryngoscope. In this regard, the screen 13 is preferably a touch screen.

In this regard, the head 40 includes cooperating magnetic elements 12 such that when the head 40 contacts the recharging base both elements are aligned and without any movement that efficiently recharge the interior battery 14. In this concern, the head 40 is designed such that its surface has a superficial area necessary for maintaining the videolaryngoscope in vertical position and supporting the total weight of the videolaryngoscope when the interior battery 14 is charging.

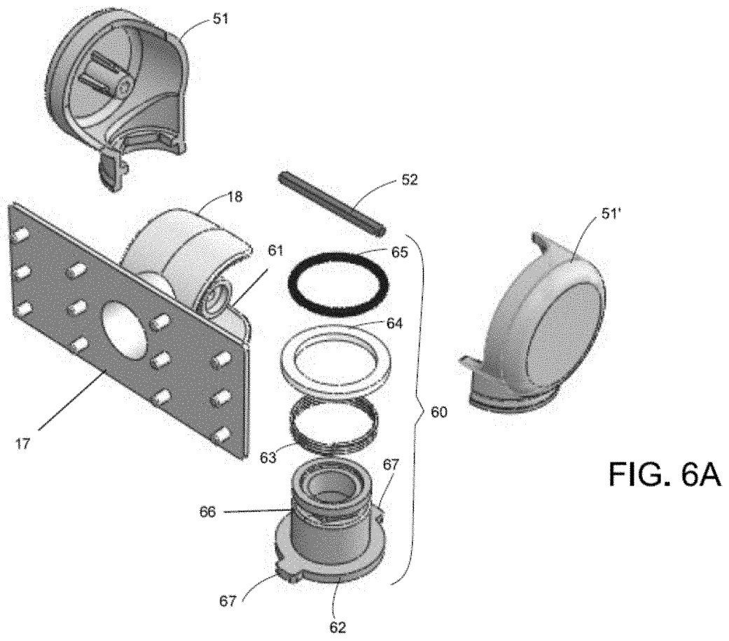

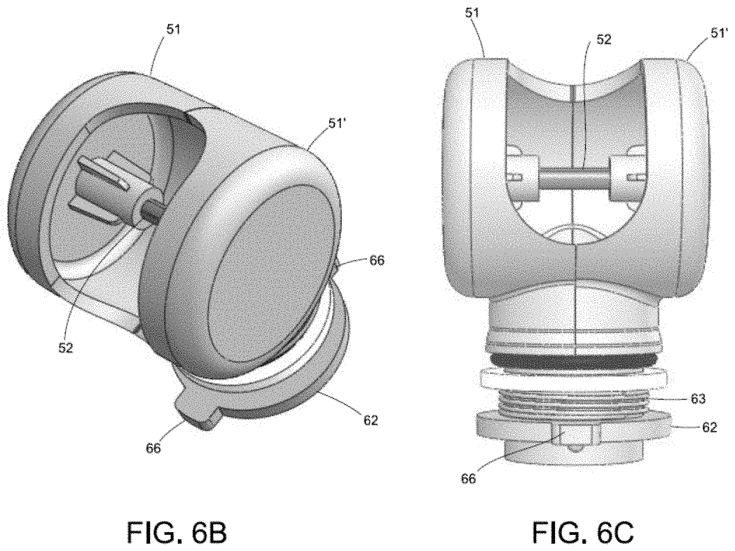

In addition, reference is made to FIGS. 4 and 5 as well as to FIGS. 6A, 6B and 6C in order to mention that the head 40 also comprises a rotatory back support 17 connected to the rear face 44, the support 17 is coupled to a mounting base 50 allowing the head 40 to upwardly or downwardly rotate relative to the handle 20.

In this regard, the mounting base 50 comprises two lateral halves 51 and 51′ in a circular manner when laterally observed and being coupled therebetween, the halves 51 and 51′ are traversed in its center via a prestressed helical axis 52. On the other hand, the back support 17 includes a coupling 18 having a “C” shape being coupled inside the lateral halves 51 and 51′ once coupled, said helical axis 52 traverses the coupling 18 and the lateral halves 51 and 51′ allowing the head 40 being pivotable in a controlled manner on the axis 52 so that the head 40 is upwardly and downwardly moved relative to the handle 20.

### AQUI

More particularly, the videolaryngoscope comprises a rotation mechanism 60 in order to allow the head 40 to rotate relative to the handle 20 in the proximal end. The rotation mechanism 60 comprises an upper support 61 coupled among the lateral halves 51 and 51′, a tubular support 62 wherein the upper support 61 is received. The tubular support 62 is fixed to the handle; other elements of the rotation mechanism are the washers 64 and 65 mounted near the upper end of the upper support for providing a sealing connection of the head relative to the handle. The tubular support 62 comprises a circumferential seat 66 for receiving a spring 63 wrapping the seat 66. The tubular support 62 comprises in its lower portion stops 66 diametral opposed and radially projecting to limit the movement of the tubular element 62 relative to the handle, and is such manner, by rotating the tubular element 62 in a direction or another, the spring is stressed regulating the rotation of the head being limited by such stops 66.

Regarding the manner in which the mounting base 30 is mounted to the handle 20 for having at least one angular position which is blocked for maintaining the mounting base 30 in a desired angle relative to the longitudinal axis of the handle 20, the following may be mentioned now referring to FIG. 4, in the preferred embodiment, the mounting base 30 is formed by two halves 33 and 33′ provided with a coupling internal element 32 and 32′ having a disk shape, inside the halves of the blade there is a pair of internal reinforcements 34 and 34′ that are located among the halves 33 and 33′, the reinforcements also have an internal portion 35 and 35′ in a disk shape. In such way, by coupling the halves 33 and 33′, the internal reinforcements 34 and 34′ are located among said halves such that the internal elements 32 and 32′ of the supports and the internal portion 35-35′ of the halves are traversed by an axis 85 provided with a transversal blocking element 87 that couples in a cooperating manner into the inserts 90 and 91. In FIG. 4, it is also observed a pressure button 81 which is connected to the axis 85 which traverses the elements 33 and 35 and is wrapped by a spring 88 and a washer 89.

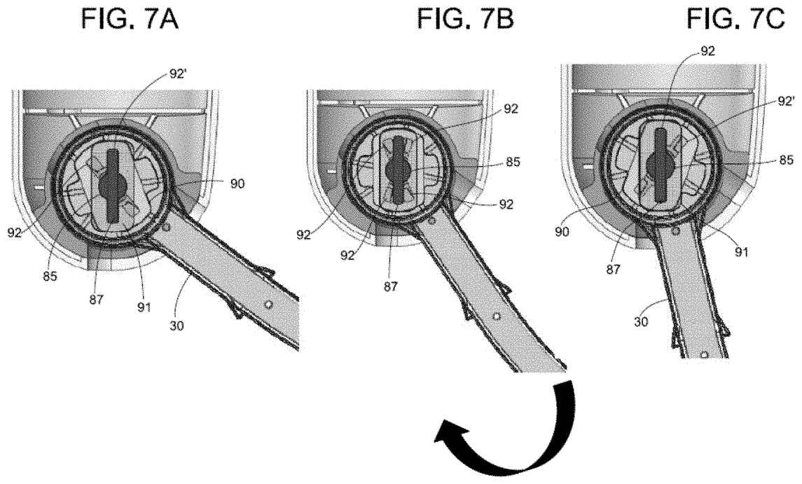

Now, with the assistance of FIGS. 7A, 7B, and 7C, it is below explained the manner in which the mounting base for blade 30 is unblocked, moved and blocked to a new position, FIG. 7A shows the blade base 30 in a blocked position since the blocking element 87 connected to the axis 85 is inserted into one of the positioning slots 92 and 92′ of the insert 90, in this sense each of the positioning slots 92 and 92′ corresponds to an angular position of the base for blade 30. In addition, the blocking element 87 is inserted into both of the inserts 90 and 91.

FIG. 7B corresponds to the moment in which the button is pressed down, thus releasing the element 87 of the positioning slot 92′ such that the blade base is free for moving the same to the desired position, by reaching the next position and stop pressing the button, then the blocking element 87 returns for being received and coupled into the slot 92 such that the insert is fixed therein as shown in FIG. 7C for once again blocking it in the new position corresponding to FIG. 7C.

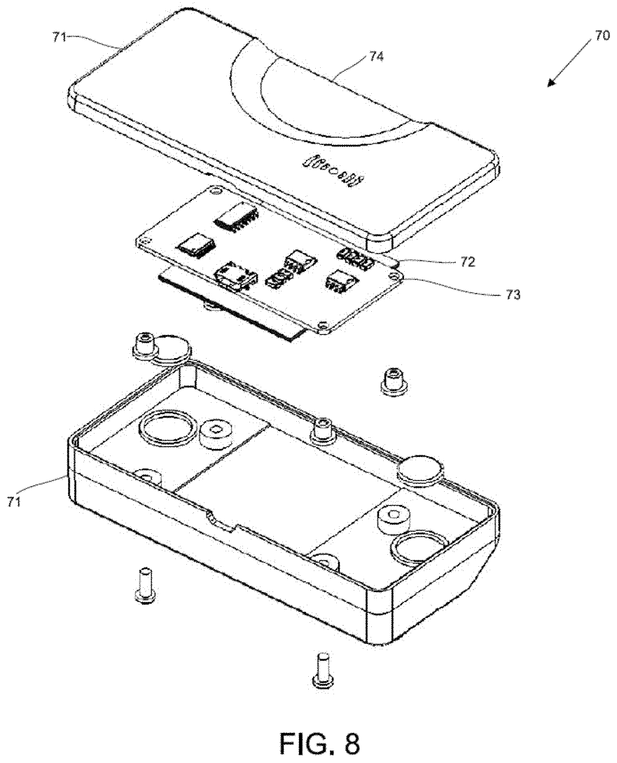

Now reference is made to FIG. 8 that shows an exploded view of the recharging base 70 comprising a housing 71 wherein the power generating coil 72 is located; the base also comprises a controller 73 inside the housing 71 and operationally connected to the coil 72 for regulating the time and charge intensity of the coil 72 and the base has a cavity 74 wherein the head is coupled.

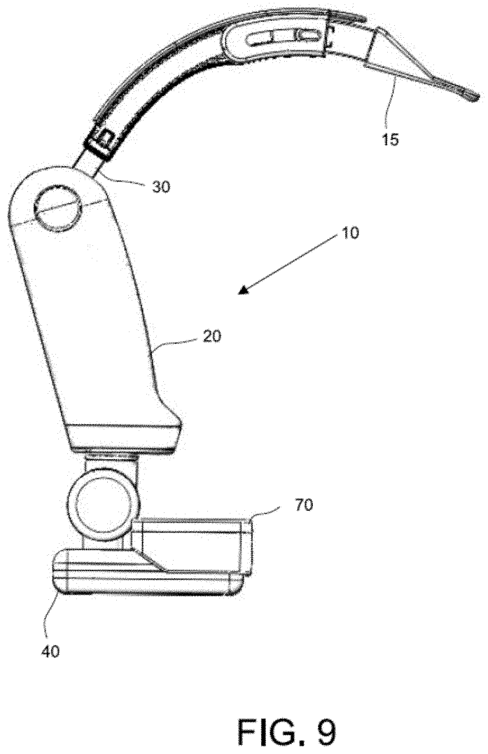

In FIG. 9, it is shown the videolaryngoscope 10 being coupled to the recharge base 70 such that as observed, the head 40 may support all the weight of the videolaryngoscope in said position, FIG. 9 additionally shows a videolaryngoscope blade 15 being mounted to the blade base 30.

According to the aforementioned, it is evident for one skilled in the art that the embodiment of the videolaryngoscope previously disclosed is presented only with illustrative purposes, since one skilled in the art may perform numerous variations thereof, when and if they are designed according to the principles of instant invention. Consequently, instant invention includes all the embodiments one skilled in the art may pose from the concepts contained in present description according to the following claims.

### REFERENCE LIST

10 Videolaryngoscope
   11 Videocamera.
   12 Magnets
   13 Screen
   14 Battery
   15 Videolaryngoscope blade
   17 Upper support
   18 Coupling element
20 Handle
   21 Proximal end
   22 Distal end
   25 Flat wall
30 Mounting base for blade
   31 Distal end
   32 Proximal end
   33 Halves
   34 Internal halves
   35 Circular ends
40 Head
   41 Coil
   42 Housing
   43 Frontal face
   44 Rear face
50 Rotatory mounting base
   51 Halves
   52 Prestressed axis
60 Rotation mechanism
   61 Upper support
   62 Tubular element
   63, 64, 65 Washers
   66 Stops
70 Recharging base
   71 Housing
   72 Generating coil
   73 Controller
   74 Cavity
80 Rotatory mechanism
   81 Button
   85 Axis
   87 Blocking element
   88 Spring
   90 Blocking insert
   91 Blocking insert
   92, 92′ Slots

What is claimed is:

1. A videolaryngoscope comprising:
a hollow fastening handle with a proximal end and a distal end;
a mounting base for a videolaryngoscope blade, being the mounting base connected to the distal end of the handle in a rotatory manner for varying its angular position relative to the handle, the mounting base having a distal end;
a pivotable button including a blocking mechanism for the mounting base of the blade, wherein the button secures or frees the rotatory mechanism of the mounting base of the videolaryngoscope blade when the button is actuated;
a videocamera mounted to the distal end of the mounting base for the videolaryngoscope blade;
a head mounted to the proximal end of the handle,
a screen housed in the head and operationally communicated to the videocamera,
an electric charge receiving system having an induction power receiving coil located in the head;
an internal rechargeable power source operationally connected to the charge receiving coil; and
an electric charge emission system having a charge base being coupled to the head and including an induction power generating coil such that when the charge base is coupled to the head, the internal power source is recharged.

2. The videolaryngoscope according to claim 1, wherein the internal power source is a battery located in the interior of the videolaryngoscope.

3. The videolaryngoscope according to claim 2, wherein the battery is located in the interior of the handle.

4. The videolaryngoscope according to claim 1, wherein the head and the recharge base include cooperating magnetic elements for maintaining the charge base coupled to the head.

5. The videolaryngoscope according to claim 1, wherein the head comprises:
a housing with a frontal face and a rear face;
wherein the screen is located in the frontal face; and the power receiving coil is located next to the rear face.

6. The videolaryngoscope of claim 5, wherein the screen is immersed relative to the frontal face.

7. The videolaryngoscope according to claim 1, wherein the screen is a touch screen.

8. The videolaryngoscope according to claim 1, wherein the head is designed in order to support and maintain in vertical position the videolaryngoscope when the internal battery is charging.

9. The videolaryngoscope according to claim 1, wherein the charge base comprises:
a housing wherein the power generating coil is located;
a controller inside the housing and operationally communicated to the coil for regulating the time and charge intensity; and
a cavity coupled to the head.

10. The videolaryngoscope according to claim 1, wherein the head is mounted to the handle in rotatory manner relative to i) the axial axis of the handle and ii) to an axis perpendicular to the handle.

11. The videolaryngoscope according to claim 1, wherein the mounting base for the laryngoscope blade has at least one angular position being blocked for maintaining the mounting base in a desired angle.

12. The videolaryngoscope according to claim 11, wherein the mounting base includes two halves being coupled therebetween, an interior end wherein a rotatory mechanism is provided being housed and mounted in the handle and a blocking element for maintaining the mounting base in the desired angle.

13. The videolaryngoscope according to claim 12, wherein the rotatory mechanism comprises a circular support in each half traversed by an axis.

14. The videolaryngoscope according to claim 13, wherein the distal tip of the mounting base for the videolaryngoscope blade provides at least one light.

15. The videolaryngoscope according to claim 1, wherein each of the elements thereof are impermeable.
